(19) United States
(12) Patent Application Publication
(10) Pub. No.: US 2024/0233931 A1
(43) Pub. Date: Jul. 11, 2024

(54) MEDICAL DEVICE LIFE CYCLE DETERMINATION AND MANAGEMENT

(71) Applicant: Stryker Corporation, Kalamazoo, MI (US)

(72) Inventors: Doan Huu Dinh, Seattle, WA (US); Ryan Peter Bowman, Richland, WA (US); Karl Frederick Gauglitz, Snohomish, WA (US)

(73) Assignee: Stryker Corporation, Kalamazoo, MI (US)

(21) Appl. No.: 18/406,001

(22) Filed: Jan. 5, 2024

Related U.S. Application Data
(60) Provisional application No. 63/437,603, filed on Jan. 6, 2023.

(51) Int. Cl. G16H 40/40; G16H 15/00

(52) U.S. Cl. CPC G16H 40/40; G16H 15/00

(57) ABSTRACT

Systems and methods described herein relate to tracking indications of use for medical device accessories to track and determine end of life for the sensors as well as to enforce an end of life for various accessories. The indications of use may be tracked by a medical device to which the accessory is connected, with the accessory uniquely identified by an identifier that the medical device may associate with the indications of use. The indications of use and identity information for the accessory may be conveyed to a cloud-based system for tracking lifespan of accessories. The medical device and/or cloud-based system may determine an end-of-life based on the specific indications of use and communicate the lifespan, expiration, or expected expiration to an owner or user of the accessory.

### CROSS-REFERENCE TO RELATED APPLICATION

This application claims priority to U.S. Provisional Application No. 63/437,603, titled “MEDICAL DEVICE LIFE CYCLE DETERMINATION AND MANAGEMENT” and filed on Jan. 6, 2023, which is incorporated by reference herein in its entirety.

### BACKGROUND

The use of field-deployed medical devices, such as portable defibrillators, is achieving widespread acceptance. Such devices are used to help provide critical medical treatment to patients as close to the time of need as possible. Because of their use in medical emergencies, such medical devices are subject to regulatory approval. Such medical devices are designed, tested, and approved as a system. However, some of the medical devices may operate using consumable components or components that have a limited life and must be replaced. Because proper operation and regulatory approval is contingent upon the system being configured properly, it may be important that such components be replaced with authorized replacements that are proper for the medical device.

### BRIEF DESCRIPTION OF THE DRAWINGS

FIG. 1 illustrates an example system for determining and managing life cycles of medical device accessories, according to at least one example.

FIG. 2 is an example block diagram of a host device and accessory illustrating determination and management of life cycle for the accessory, according to at least one example.

FIG. 3 is an example block diagram of a system for monitoring life cycles of medical device accessories, according to at least one example.

FIG. 4 is an example flowchart illustrating a method for determining and managing life cycles of medical device accessories, according to at least one example.

FIG. 5 illustrates an example of an external defibrillator configured to perform various functions described herein, according to at least one example.

FIG. 6 illustrates an example of a capnography device configured to perform various functions described herein, according to at least one example.

### DETAILED DESCRIPTION

Various implementations described herein relate to a system and method for managing life cycles of medical accessories. A method for managing life cycles of medical device accessories by associating a unique identifier of the accessory, such as from a memory chip of the medical device with a log of indications of use of the medical device accessory. The method may include identifying the unique identifier from the memory chip and storing the identifier in a memory of the medical device as well as storing at a central storage location (e.g., a cloud-based system). The indications of use of the accessory are recorded by the medical device (e.g., duration of use, number of activations, type of use, etc.) and stored in the memory of the medical device as well as uploaded to the central storage location. The indications of use, such as a use counter, stored at the central storage location may then be updated with indications of use from any medical devices that use the accessory, providing a reliable and accurate use counter for medical device accessories that is cross-device and platform.

The implementations described herein also relate to a system for replacing medical accessories at or near the end of their life cycle. The system includes one or more medical devices and one or more accessories that connect to the medical devices and include unique identifiers readable by the medical devices. The unique identifier is determined by the medical devices and uses of the accessory are logged by the medical devices and reported or stored at a central storage location. The system enables automatic re-ordering or replacement of accessories as the true end of life for the accessory approaches. The true end of life may be based on an age of the accessory a use counter stored at the central storage location and may be adjustable based on data gathered from medical device accessories indicative of the true end of life, e.g., number of uses, type of uses, duration of use, age of accessory, etc. before failure of the accessory occurs. In this manner, the true end of life may be based on real-life uses rather than a set date for expiration that may not consider indications of use and amount of use before reaching the set date.

In some examples, medical device manufacturers may define product requirements for reliability of medical devices and accessories. The product requirements may define, for example, how many cycles of use a product should last before being replaced. The product requirements may be based on rough estimates, since use data is often difficult to accurately collect using conventional systems. Furthermore, although medical device manufacturers recommend that their accessories be replaced after a certain number of years, they often have difficulty in enforcing their customers to do so. Additionally, even if a customer were to replace an accessory at the recommended time, they might be retiring a still-good product if they did not use it as many times as the manufacturer estimated. In some examples however, the customer might be retiring an extremely worn-out product that should have been retired long before the recommended time if they used it much more than the manufacturer estimated or anticipated. To prevent this latter situation, users should be replacing their medical device accessories after a certain number of uses instead of only a certain timeframe. Thus, there is a benefit to a medical device manufacturer having both accurate use data and a way to ensure their customers replace their accessories at the end of their true life cycles. By embedding memory chips in an accessory, a manufacturer can store information regarding that accessory to be read by a medical device when connected to the medical device.

In some examples, an electrically erasable programmable read-only memory (EEPROM) memory device may be built-in to accessories and may be used for authentication and/or identification of the accessories when connected to a medical device. These EEPROMs may be read-only protected after initial programming, and thus a use counter stored on the accessory's memory chip may require constant rewriting of the chip. In some examples however, the contents of the read-only chip may be associated with a use counter that is stored at a memory of the medical device and/or a central storage location (e.g., a cloud-based storage system).

In an illustrative example, an accessory's memory chip stores a unique identifier that is unique to that specific accessory, e.g., its serial number. Once the accessory is connected to a medical device and has the contents of the memory chip read, the medical device can store the unique identifier in its memory and associate that with indications of use. The indications of use could include a variety of information, numbers of insertions, removals, therapies delivered, duration of physiological monitoring, etc. In the illustrative example, a customer may connect a brand-new therapy cable (with serial number xyz) to a defibrillator A and a patient simulator for the first time. After 5 minutes of monitoring using the patient simulator, the customer may then disconnect it from the defibrillator. In this case, defibrillator A may store data in its prognostic log (its history log) that shows that therapy cable xyz was inserted once, removed once, and used for 5 minutes of monitoring. At a second time, later than the initial use, the customer inserts the same therapy cable xyz into defibrillator A again and this time delivers a defibrillation pulse into a test load before removing the therapy cable. Now, the defibrillator A may update its prognostic log to show that therapy cable xyz was inserted twice, removed twice, used for 5 minutes of monitoring, and defibrillated once into a test load.

Additionally, the medical device's memory of accessory use can be uploaded to a cloud-based service that can be accessed and updated (through summation of use counters and times) by other medical devices. Then, an accessory used with the first medical device will have its use counter updated by a second medical device should it be used with that, and so on. In the illustrative example, the defibrillator may be connected to a data service. The use data for therapy cable xyz would be uploaded to the data service. When the therapy cable xyz is inserted in and then removed from a second defibrillator B, defibrillator B would upload this additional use data to the data service, where therapy cable xyz's use data would then be updated to show that it has been inserted 3×, removed 3×, used for 5 minutes of monitoring, and defibrillated once.

The data collected from the medical devices may then then be used to help enforce the replacement of worn-out accessories. When an accessory nears its true end of life, a field service representative can be informed so they can contact the customer to replace the accessory. The data can also be used to generate a message on the medical device or send an e-mail to the customer to inform them directly. In some examples, the data can be used to generate an order and/or order information to automatically re-order or provide budgets for replacement accessories for the customer.

In some examples, the use data collected for accessories may be used to determine and refine use cycles and thresholds for use for accessories. As used herein, the term “use cycle,” and its equivalents, may refer to an event in an accessory's lifetime in which the accessory is utilized for monitoring or treatment. For instance, a use cycle may begin when the accessory is activated or coupled with a host device, and the use cycle may end when the accessory is deactivated or decoupled from the host device. The predetermined threshold may include various indications of use such as thresholds for use cycles, quantity or volume of data or matter conveyed by the accessory, use time, and other such use data. The predetermined threshold may comprise an aggregate use threshold that may be determined based on a combination of one or more different types of use data, for example based on number of connections as well as a charge volume delivered by defibrillator leads. This use data and the associated threshold may help reduce the number of field failures due to worn-out accessories. Additionally, the use data may be used to ensure that accessories are replaced and to enforce the true end-of-life for the accessories to improve device performance and ensure the medical device acts according to intended purposes.

Various implementations described herein are directed to specific improvements in the technical field of emergency medical devices. For instance, by improving accuracy and enforcing true end-of-life for accessories, the medical devices may be used with confidence.

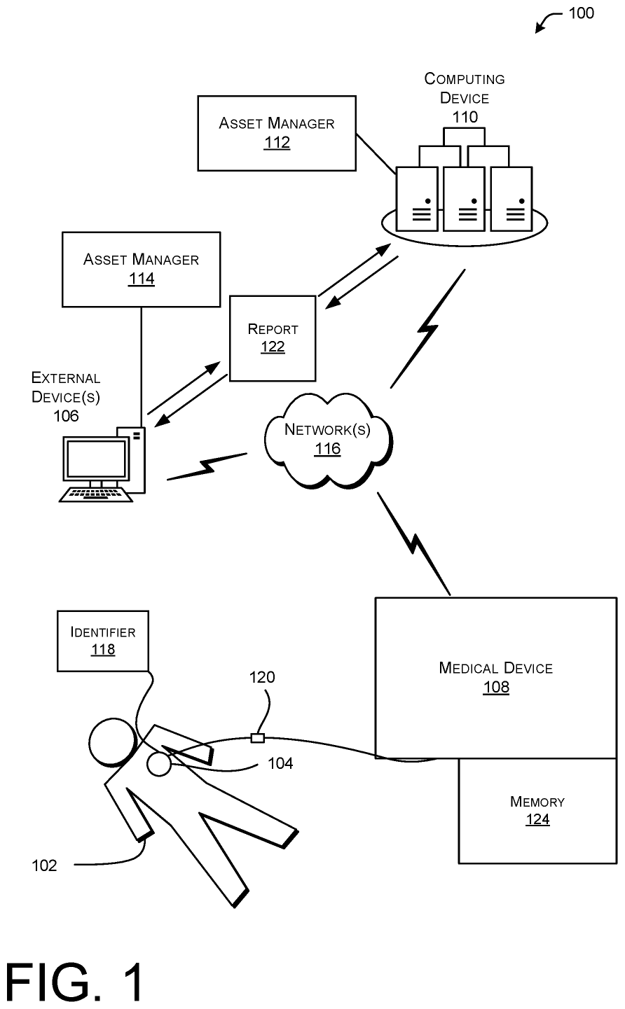

FIG. 1 illustrates a system 100 for determining and managing use data for medical device accessories, according to at least one example. In some implementations, the system 100 includes an accessory 104 configured to detect sensor data or perform a therapy for a patient 102. In some implementations, the accessory 104 includes a variety of different sensors, including respiration sensors, cardiac sensors, oxygenation sensors, capnography sensors, and other such devices configured to detect one or more physiological parameters of the patient 102. In some examples the physiological parameter includes electrocardiogram (ECG) data or other non-ECG data (e.g., a heart rate level or waveform, a temperature level or waveform, an airway CO₂ level or waveform, an oxygenation level or waveform, a blood pressure level or waveform, etc.). In some examples, the accessory 104 may include leads for a defibrillator, tubes, passages, valves, end-effectors, or other components for providing a therapy to the patient 102 from a medical device 108. The medical device 108 may, in some examples include a defibrillator (e.g., an Automated External Defibrillator (AED) or a monitor-defibrillator), a mechanical chest compression device, a ventilation device, a capnography device, or other such treatment device.

The accessory 104 includes an identifier 118 that may be read by the medical device 108. The identifier 118 may include a physical device that conveys data to the medical device 108. The identifier 118 may also include a representation of a serial number, for example represented as a barcode, quick-read (QR) code, or other visible marker that may be scanned or read by the medical device 108. The identifier 118 may further include data stored on a memory device and/or other device such as an RFID tag coupled to the accessory, such as a memory chip that includes a stored serial number and/or an RFID tag encoded with the serial number. In some examples, the identifier 118 may include a memory device storing information such as an identifier, a visual identifier such as a quick response (QR) code, barcode, or other such optical identifier, and may also include other identifiers used to uniquely identify the accessory 104, such as an RFID chip or other such component.

The medical device 108 includes a memory 124 that may store information such as the identifier 118 and use data for the accessory 104. As used herein, the term “use data,” and its equivalents, may refer to data indicating an extent to which an accessory has been utilized. The indications of use of the accessory are recorded by the medical device (e.g., duration of use, number of activations, type of use, etc.) and stored in the memory of the medical device as well as uploaded to the central storage location. The memory 124 may store the identifier information and the indications of use in a table that may be updated with additional uses. In some examples, the accessory 104 may include a memory device that stores one or more indications of use in addition to the unique identifier. The indications of use may be communicated to the medical device 108.

The medical device 108 may communicate with the accessory 104 through a communication channel that includes a fusable link 120 that may be interrupted based on a signal from the medical device 108. For example, the asset manager 112 may determine that the accessory 104 has reached its end of life based on the uses logged for the accessory 104. The asset manager 112 may communicate the end of life to the medical device 108. The medical device 108 may send a signal to the fusable link 120 to break the communication channel between the medical device 108 and the accessory 104, and thereby prevent further use of the accessory after the end of life, as determined by the asset manager. In some examples, such a system may also be used with authentication protocols, for example to prevent the use of unauthorized systems or protocols through the medical device 108.

In some examples, the medical device 108 is configured to communicate over a network 116, which includes wired and/or wireless connections, to an external device 106, such as a computing device having a processor and processor executable instructions to perform various actions, and a computing device 110, which may include an asset manager 112. The asset manager 112 may be a cloud-based service for managing and tracking indications of use for the accessory 104 based on data uploaded from the medical device 108. In some examples, the external device 106, which may include a client device, such as a system of a service provider associated with the medical device 108. The asset manager 112 may be embodied on the computing device 110 and/or the external device 106.

The computing device 110 and/or the external device 106 may generate report data 122 through the asset manager 112 and/or the asset manager 114. The report data 122 may include indications of use as logged by the medical device 108 and communicated over the network 116. The report data 122 may include information such as accessories and/or medical devices approaching and/or at their end-of-life (as determined by the asset manager 112). The report data 122 may include order information such as accessories and/or medical devices to re-order or replace based on the determined end-of-life.

In some examples, the asset manager 112 and/or asset manager 114 may use one or more machine learning systems and/or neural networks to determine the life cycle of devices based on use data from an array of medical devices and/or accessories over a network associated with the computing device 110 and/or external device 106. The medical device 108 and/or other components of the external device 106 and/or computing device 110 may perform one or more operations as described herein in some examples.

Although discussed in the context of neural networks, any type of machine-learning can be used consistent with this disclosure. For example, machine-learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet-50, ResNet-101, VGG, DenseNet, PointNet, and the like. In some examples, the ML model discussed herein may include PointPillars, SECOND, top-down feature layers, and/or VoxelNet. Architecture latency optimizations may include MobilenetV2, Shufflenet, Channelnet, Peleenet, and/or the like. The ML model may include a residual block such as Pixor, in some examples.

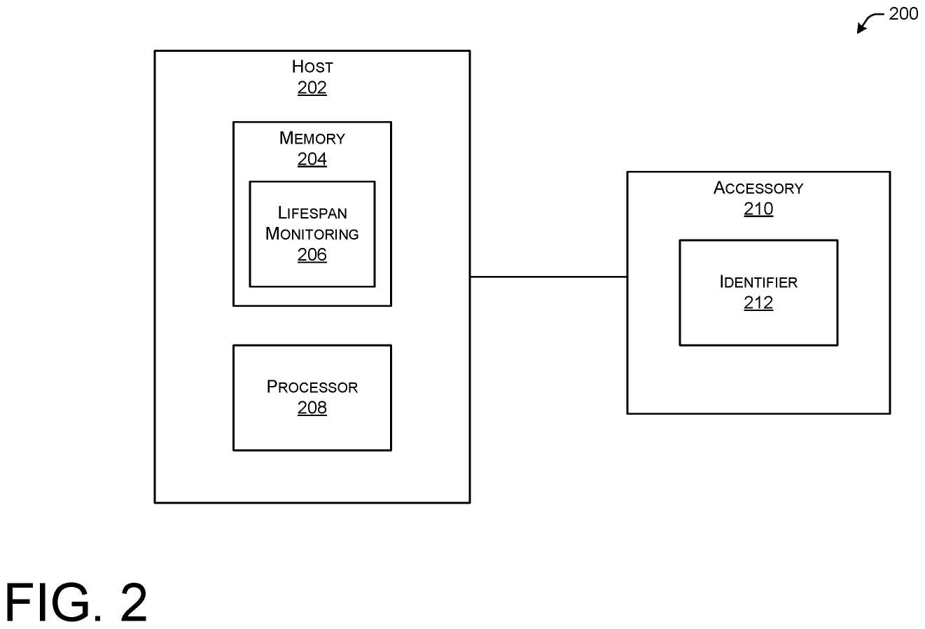

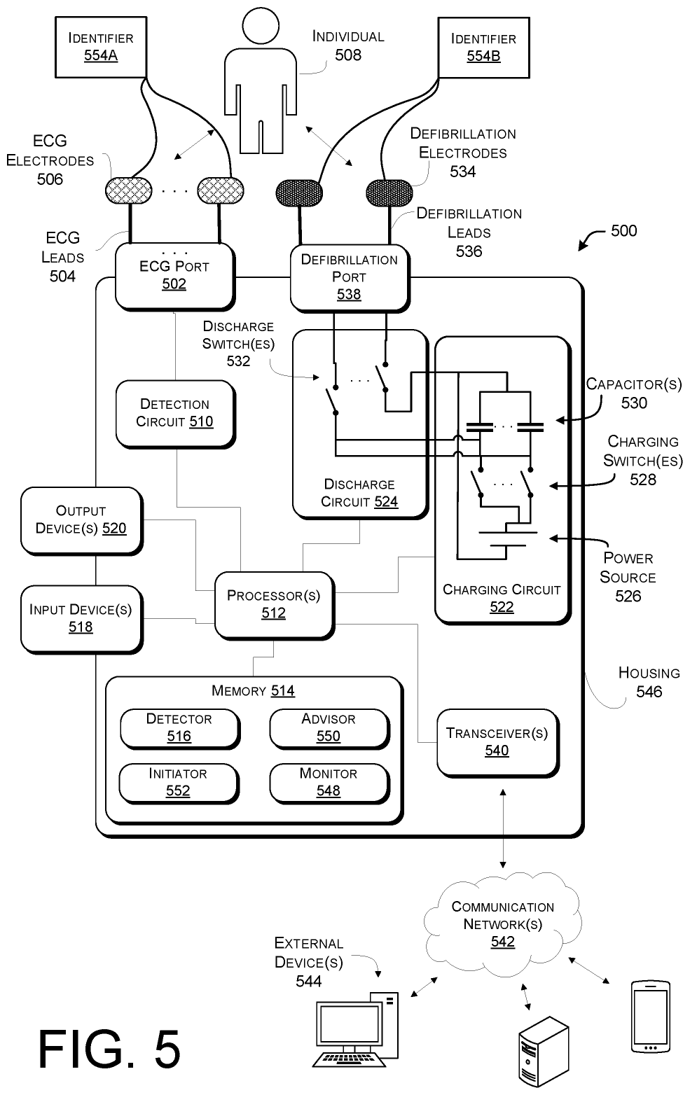

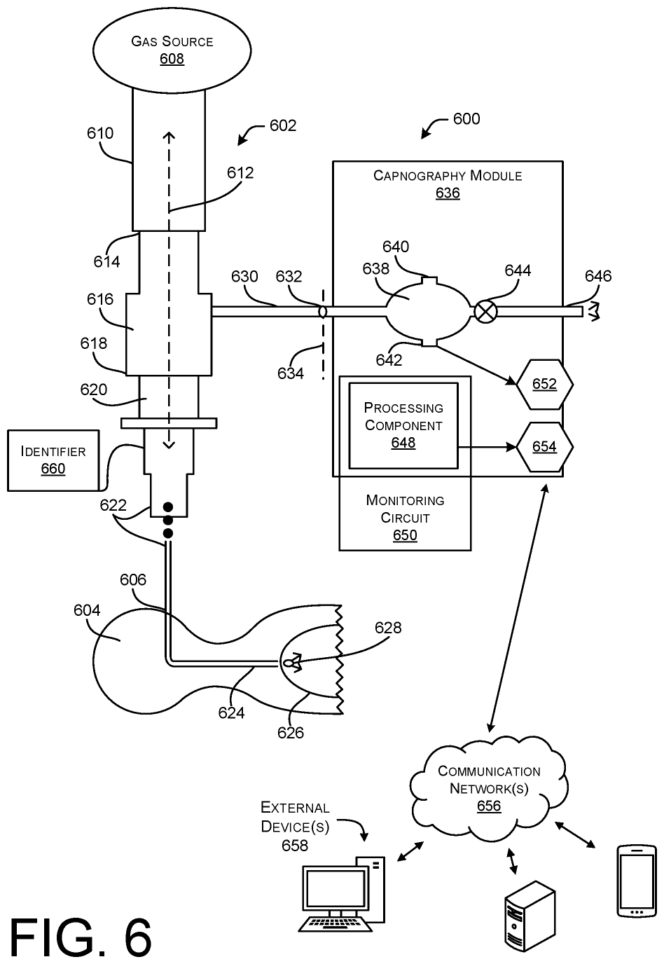

FIG. 2 is an example block diagram 200 of a host device 202 and accessory 210 illustrating determination and management of life cycle for the accessory, according to at least one example. Examples of accessory 210 include an electrode, probe, a component of a point-of-care diagnostic device such as test leads, electrodes, or other such components for diagnostics of a patient, video laryngoscope, or another device which may be connected to a medical device. Examples of a host device 202 include a defibrillator, a monitor, CPR assist device, or the like, such as described with respect to FIGS. 5 and 6 herein. The accessory attachment to the host device 202 may be either wired or non-wired. For example, in the case of a non-powered accessory 210, the accessory attachment may be wired. In some non-wired attachments, the accessory 210 may be self-powered. In this implementation, the accessory 210 is associated with an identifier 212, such as identifier 118 described above with respect to FIG. 1, and may include one or more other components, such as a memory and/or processor (such as a microprocessor). The identifier 212 may include one or more unique identification systems such as visual identifiers, serial numbers, RFID tags, or other such methods and systems for storing a unique identifier (such as a serial number) for access by the host device 202.

The host device 202 includes a memory 204 and a processor 208. In some examples, the processor 208 and/or memory are used by one or more components of the host device 202 to perform the normal operations of host device 202. For instance, processor 208 retrieves and stores information from and to the memory 204 in furtherance of the operation of the host device 202.

The memory 204 includes a lifespan monitoring component 206. The lifespan monitoring component 206 may access data from the accessory 210 such as the identifier 212 and/or use data from one or more sensors of the host device 202 and/or the accessory 210. In this manner, the lifespan monitoring component 206 may log use data such as connection counts to the host device 202, time in use, charge, or volume of delivered product (e.g., charge, fluid, drug, etc.), and other such use data. The lifespan monitoring component 206 may further communicate with a cloud-based management system, such as the asset manager 112 to update and maintain an accurate use log for each individual accessory connected to one or more medical devices that report to the asset manager.

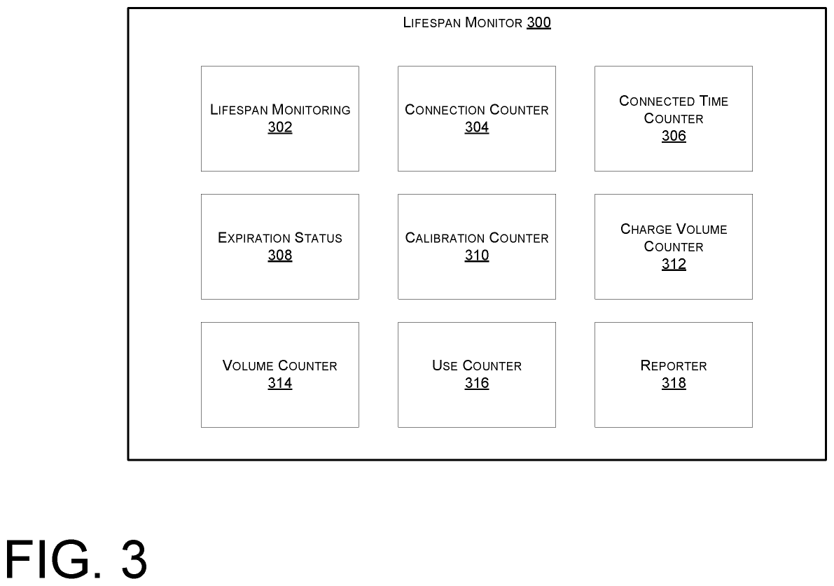

FIG. 3 is an example block diagram of a system for monitoring life cycles of medical device accessories, according to at least one example. The block diagram includes a lifespan monitor 300 that may be similar and/or identical to the lifespan monitoring component 206 of FIG. 2. In some examples, the lifespan monitor may be embodied in a memory device. The memory device may include a read only section and a read/write section. One or more of the components of the lifespan monitor 300 may be embodied in the read only and/or the read/write section.

One of ordinary skill in the art will also understand that the read only section and the read/write section can include multiple separate physical memory devices or a single memory device. The read only section may store information such as sensor life expiration limits, sensor type data, and other such data that is either largely or entirely unaffected by the use data from the accessory.

The read/write section may include a number of components for tracking the use of the accessory. For example, the lifespan monitor 300 includes a connection counter 304 for monitoring numbers of times the accessories are connected to medical devices (which may be determined by the medical device based on identifying the identify of a connected accessory and/or by a sensor of an accessory used to detect connections with medical devices, e.g., a voltage or current sensor or other sensor configured to detect the physical connection to the medical device), a connected time counter 306 for monitoring the amount of time (e.g., elapsed time) while the accessory is connected and/or active), an expiration status 308 (e.g., a binary expired or non-expired for the accessory), a calibration counter 310 for logging a number of times the accessory has been calibrated and/or whether the device is maintaining operation within a calibration range, a charge volume counter 312 that may log a volume or quantity of charges applied through an accessory such as a defibrillator, a volume counter 314 for gas through a capnography sensor or other such device, a user counter 316 that may capture one or more additional indications of use. As used herein, the term “charges,” and its equivalents, may refer to a process by which at least one capacitor is charged with electrical energy. For example, a charge is delivered when the capacitor(s) discharge the stored electrical energy. In some instances, a charge is delivered as an electrical shock (e.g., a defibrillation treatment) or a pace pulse. Although described in relation to certain parameters and information, a person of ordinary skill in the art will understand from the disclosure herein that more or fewer read only and parameters can be stored on the memory as is advantageous in determining the useful life of a sensor.

The lifespan monitor 300 include a lifespan monitoring component 302 that may include one or more models and/or systems for determining a useful life remaining, time until end-of-life, amount of use, or other such indications based on data from the medical device and/or the accessory. As described herein, the lifespan monitoring component 302 may include one or more machine learning models for determining the lifespan and expired life of the accessory based on the uses logged by the medical device.

The lifespan monitor 300 also includes a reporter 318 that may communicate the various use parameters, identifier information, and/or data from the lifespan monitoring component 302 to an asset manager on a remote computing device.

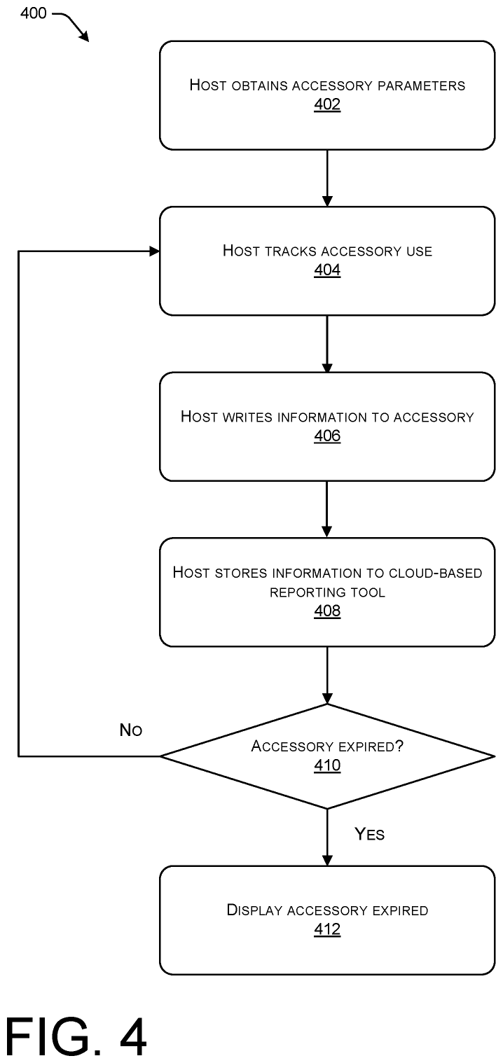

FIG. 4 illustrates a flow diagram of a method according to the present technology. For simplicity of explanation, the methods are depicted and described as a series of acts. However, acts in accordance with this disclosure can occur in various orders and/or concurrently, and with other acts not presented and described herein. Furthermore, not all illustrated acts are necessary to implement the methods in accordance with the disclosed subject matter. In addition, those skilled in the art will understand and appreciate that the methods could alternatively be represented as a series of interrelated states via a state diagram or events. Additionally, it should be appreciated that the methods disclosed in this specification are capable of being stored on an article of manufacture to facilitate transporting and transferring such methods to computing devices. The term article of manufacture, as used herein, is intended to encompass a computer program accessible from any computer-readable device or storage media.

Any of a variety of other process implementations which would occur to one of ordinary skill in the art, including but not limited to variations or modifications to the process implementations described herein, are also considered to be within the scope of this disclosure.

FIG. 4 is an example flowchart illustrating a method 400 for determining and managing life cycles of medical device accessories, according to at least one example. At 402, the method 400 includes obtaining one or more accessory parameters. The accessory parameters may include unique identifiers associated with the accessory, for example by scanning a visual identifier, reading a memory chip, accessing a stored identifier, reading an RFID tag, or otherwise accessing an identifier of the accessory. In some examples, the accessory parameters may include previous indications of use. The previous indications of use may have been written on a memory device of the accessory by a medical device to which the accessory was previously connected. In this manner, the uses by a medical device that may not connect to a cloud-based system may still be logged and maintained with the accessory to identify the true end of life of the accessory. In some examples, the parameters may also include information related to the type of accessory, end of life limits, calibration data, and other such information.

At 404, the method includes the host device (e.g., medical device) tracking accessory use during operation. For example, the host device tracks sensor use information, such as, for example, the amount of time the sensor is in use, the amount of time the sensor is connected to a finger, the number of times the sensor opens and closes, the average temperature, the average current provided to the sensor, as well as any other stress that can be experienced by the sensor. The host device may also log information for therapy providing devices such as defibrillator pads and data associated therewith such as charges delivered, numbers of connections to a defibrillator, etc. In some examples, at 406, the host device may write this use information on a periodic basis to the accessory. In such examples, the accessory may be equipped with a memory device for maintaining a local copy of the use data as determined by the host device.

At 408, the method includes storing the use information at a cloud-based reporting tool. The cloud-based reporting tool may include the asset manager 112 of FIG. 1. The cloud-based reporting tool may include a cloud-based service configured to communicate with one or more additional medical devices and one or more computing devices of a service provider. The cloud-based reporting tool may be configured to generate a report of medical devices and accessories for the service provider, the report may include expected expirations for the medical devices or accessories. The cloud-based reporting tool may be configured to generate order data may include item quantities and prices for accessories based at least in part on the expected expiration. The order data may include item quantities and prices for a predetermined time period. The cloud-based reporting tool may be configured to generate an order for submission with a supplier to replace one or more accessories based on the expected expiration. The cloud-based reporting tool may be configured to generate a message for an account associated with the medical device, the message may include the expected expiration and generated in response to the expected expiration being within the predetermined threshold period of time.

At 410, the method 400 may include determining whether or not the accessory is expired based on the data stored at the cloud-based reporting tool and/or from the host device. If the accessory's life has not expired, then the method 400 returns to block 404 where the host device continues to track use information. If, however, at decision block 410 the host device decides that the life has expired, the host device will display a message that the accessory life expired at block 412.

The accessory use information can be determined in any number of ways. For example, in an implementation, in order to determine the life of the emitters, the number of emitter pulses can be counted and an indication stored in the reporting tool by the medical device. In an implementation, the time period in which power is provided to the accessory is determined and an indication stored in the memory as well. In an implementation, the amount of current supplied to the accessory and/or LEDs is monitored and an indication is stored. In an implementation, the number of times the accessory is powered up or powered down is monitored and an indication is stored. In an implementation, the number of times the accessory is connected to a monitor is tracked and an indication is stored. In an implementation, the number of times the accessory is placed on or removed from a patient is monitored and an indication is stored. The number of times the accessory is placed on or removed from a patient can be monitored by monitoring the number of probe off conditions sensed, or it can be monitored by placing a separate monitoring device on the accessory to determine when the clip is depressed, opened, removed, replaced, attached, etc. In an implementation, the average operating temperature of the accessory is monitored and an indication stored. In an implementation, the number of times the accessory is calibrated is monitored, and an indication is stored. In an implementation, the number of patients which use an accessory is monitored and an indication is stored. This can be done by, for example, by storing sensed or manually entered information about the patient and comparing the information to new information obtained when the accessory is powered up, disconnected and/or reconnected, or at other significant events or periodically to determine if the sensor is connected to the same patient or a new patient. In an implementation, a user is requested to enter information about the patient that is then stored y and used to determine the useful sensor life. In an implementation, a user is requested to enter information about cleaning and sterilization of the accessory, and an indication is stored. Although described with respect to measuring certain parameters in certain ways, a person of ordinary skill in the art will understand from the disclosure herein that various electrical or mechanical measurement can be used to determine any useful parameter in measuring the useful life of an accessory.

The host device and/or the cloud-based system determines the accessory life based on use information. In an implementation, the systems use a formula supplied by the memory to measure the life using the above-described variables. In an implementation, experimental or empirical data is used to determine the formula used to determine the accessory's life. In an implementation, damaged and/or used accessories are examined and use information is obtained in order to develop formulas useful in predicting the useful accessory life. As described herein, a machine learning model trained using use data labeled with indications of remaining life or end of life may be used to determine the end of life for the accessories.

In an implementation, when the useful life of an accessory has been reached, the medical device or accessory may sound an alarm or gives a visual indication that the accessory is at the end of its life. In some implementations, the cloud-based system may generate a notification when an accessory is at or near the end of life for communicating to a managing device or system associated with the owner or operator of the medical device. In an implementation, the medical device will give an indication that the sensor is bad. In an implementation, an indication of the end of the accessory life is not given while the accessory is actively measuring vital signs. In an implementation, the percent of life left in an accessory is indicated. In an implementation, an estimated remaining use time is indicated. In an implementation, an indication that the end of the accessory life is approaching is indicated without giving a specific percentage or time period.

FIG. 5 illustrates an example of an external defibrillator 500 configured to perform various functions described herein. For example, the external defibrillator 500 is the medical device 108 described above with reference to FIG. 1 and/or the host device 202 described above with respect to FIG. 2.

The external defibrillator 500 includes an electrocardiogram (ECG) port 502 connected to multiple ECG leads 504. In some cases, the ECG leads 504 are removeable from the ECG port 502. For instance, the ECG leads 504 are plugged into the ECG port 502. The ECG leads 504 are connected to ECG electrodes 506, respectively. In various implementations, the ECG electrodes 506 are disposed on different locations on an individual 508. A detection circuit 510 is configured to detect relative voltages between the ECG electrodes 506. These voltages are indicative of the electrical activity of the heart of the individual 508.

In various implementations, the ECG electrodes 506 are in contact with the different locations on the skin of the individual 508. In some examples, a first one of the ECG electrodes 506 is placed on the skin between the heart and right arm of the individual 508, a second one of the ECG electrodes 506 is placed on the skin between the heart and left arm of the individual 508, and a third one of the ECG electrodes 506 is placed on the skin between the heart and a leg (either the left leg or the right leg) of the individual 508. In these examples, the detection circuit 510 is configured to measure the relative voltages between the first, second, and third ECG electrodes 506. Respective pairings of the ECG electrodes 506 are referred to as “leads,” and the voltages between the pairs of ECG electrodes 506 are known as “lead voltages.” In some examples, more than three ECG electrodes 506 are included, such that 5-lead or 12-lead ECG signals are detected by the detection circuit 510.

The ECG electrodes 506 may include identifier data 554A that may include unique identifiers for the different electrodes that may be used to uniquely track the uses for each of the ECG electrodes and thereby determine the useful life and/or end of life for each ECG electrode as described herein.

The detection circuit 510 includes at least one analog circuit, at least one digital circuit, or a combination thereof. The detection circuit 510 receives the analog electrical signals from the ECG electrodes 506, via the ECG port 502 and the ECG leads 504. In some cases, the detection circuit 510 includes one or more analog filters configured to filter noise and/or artifact from the electrical signals. The detection circuit 510 includes an analog-to-digital (ADC) in various examples. The detection circuit 510 generates a digital signal indicative of the analog electrical signals from the ECG electrodes 506. This digital signal can be referred to as an “ECG signal” or an “ECG.”

In some cases, the detection circuit 510 further detects an electrical impedance between at least one pair of the ECG electrodes 506. For example, the detection circuit 510 includes, or otherwise controls, a power source that applies a known voltage (or current) across a pair of the ECG electrodes 506 and detects a resultant current (or voltage) between the pair of the ECG electrodes 506. The impedance is generated based on the applied signal (voltage or current) and the resultant signal (current or voltage). In various cases, the impedance corresponds to respiration of the individual 508, chest compressions performed on the individual 508, and other physiological states of the individual 508. In various examples, the detection circuit 510 includes one or more analog filters configured to filter noise and/or artifact from the resultant signal. The detection circuit 510 generates a digital signal indicative of the impedance using an ADC. This digital signal can be referred to as an “impedance signal” or an “impedance.”

The detection circuit 510 provides the ECG signal and/or the impedance signal one or more processors 512 in the external defibrillator 500. In some implementations, the processor(s) 512 includes a central processing unit (CPU), a graphics processing unit (GPU), both CPU and GPU, or other processing unit or component known in the art.

The processor(s) 512 is operably connected to memory 514. In various implementations, the memory 514 is volatile (such as random-access memory (RAM)), non-volatile (such as read only memory (ROM), flash memory, etc.) or some combination of the two. The memory 514 stores instructions that, when executed by the processor(s) 512, causes the processor(s) 512 to perform various operations. In various examples, the memory 514 stores methods, threads, processes, applications, objects, modules, any other sort of executable instruction, or a combination thereof. In some cases, the memory 514 stores files, databases, or a combination thereof. In some examples, the memory 514 includes, but is not limited to, RAM, ROM, electrically erasable programmable read-only memory (EEPROM), flash memory, or any other memory technology. In some examples, the memory 514 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information, and which can be accessed by the processor(s) 512 and/or the external defibrillator 500. In some cases, the memory 514 at least temporarily stores the ECG signal and/or the impedance signal.

In various examples, the memory 514 includes a detector 516, which causes the processor(s) 512 to determine, based on the ECG signal and/or the impedance signal, whether the individual 508 is exhibiting a particular heart rhythm. For instance, the processor(s) 512 determines whether the individual 508 is experiencing a shockable rhythm that is treatable by defibrillation. Examples of shockable rhythms include ventricular fibrillation (VF) and ventricular tachycardia (V-Tach). In some examples, the processor(s) 512 determines whether any of a variety of different rhythms (e.g., asystole, sinus rhythm, atrial fibrillation (AF), etc.) are present in the ECG signal.

The processor(s) 512 is operably connected to one or more input devices 518 and one or more output devices 520. Collectively, the input device(s) 518 and the output device(s) 520 function as an interface between a user and the defibrillator 500. The input device(s) 518 is configured to receive an input from a user and includes at least one of a keypad, a cursor control, a touch-sensitive display, a voice input device (e.g., a speaker), a haptic feedback device, or any combination thereof. The output device(s) 520 includes at least one of a display, a speaker, a haptic output device, a printer, or any combination thereof. In various examples, the processor(s) 512 causes a display among the input device(s) 518 to visually output a waveform of the ECG signal and/or the impedance signal. In some implementations, the input device(s) 518 includes one or more touch sensors, the output device(s) 520 includes a display screen, and the touch sensor(s) are integrated with the display screen. Thus, in some cases, the external defibrillator 500 includes a touchscreen configured to receive user input signal(s) and visually output physiological parameters, such as the ECG signal and/or the impedance signal.

In some examples, the memory 514 includes an advisor 550, which, when executed by the processor(s) 512, causes the processor(s) 512 to generate advice and/or control the output device(s) 520 to output the advice to a user (e.g., a rescuer). In some examples, the processor(s) 512 provides, or causes the output device(s) 520 to provide, an instruction to perform CPR on the individual 508. In some cases, the processor(s) 512 evaluates, based on the ECG signal, the impedance signal, or other physiological parameters, CPR being performed on the individual 508 and causes the output device(s) 520 to provide feedback about the CPR in the instruction. According to some examples, the processor(s) 512, upon identifying that a shockable rhythm is present in the ECG signal, causes the output device(s) 520 to output an instruction and/or recommendation to administer a defibrillation shock to the individual 508.

The memory 514 also includes an initiator 552 which, when executed by the processor(s) 512, causes the processor(s) 512 to control other elements of the external defibrillator 500 in order to administer a defibrillation shock to the individual 508. In some examples, the processor(s) 512 executing the initiator 552 selectively causes the administration of the defibrillation shock based on determining that the individual 508 is exhibiting the shockable rhythm and/or based on an input from a user (received, e.g., by the input device(s) 518. In some cases, the processor(s) 512 causes the defibrillation shock to be output at a particular time, which is determined by the processor(s) 512 based on the ECG signal and/or the impedance signal.

The memory 514 includes a monitor 548 which, when executed by the processor(s) 512, causes the processor(s) to determine reliability index information and/or determine display settings for data to be displayed on output device(s) 520 and/or external devices 544. The monitor 548 includes the components and/or be configured to perform the functions of the monitoring component 206, monitoring module 222, and/or monitoring component 302 described with respect to FIGS. 2 and 3.

The processor(s) 512 is operably connected to a charging circuit 522 and a discharge circuit 524. In various implementations, the charging circuit 522 includes a power source 526, one or more charging switches 528, and one or more capacitors 530. The power source 526 includes, for instance, a battery. The processor(s) 512 initiates a defibrillation shock by causing the power source 526 to charge at least one capacitor among the capacitor(s) 530. For example, the processor(s) 512 activates at least one of the charging switch(es) 528 in the charging circuit 522 to complete a first circuit connecting the power source 526 and the capacitor to be charged. Then, the processor(s) 512 causes the discharge circuit 524 to discharge energy stored in the charged capacitor across a pair of defibrillation electrodes 534, which are in contact with the individual 508. For example, the processor(s) 512 deactivates the charging switch(es) 528 completing the first circuit between the capacitor(s) 530 and the power source 526, and activates one or more discharge switches 532 completing a second circuit connecting the charged capacitor 530 and at least a portion of the individual 508 disposed between defibrillation electrodes 534.

The energy is discharged from the defibrillation electrodes 534 in the form of a defibrillation shock. For example, the defibrillation electrodes 534 are connected to the skin of the individual 508 and located at positions on different sides of the heart of the individual 508, such that the defibrillation shock is applied across the heart of the individual 508. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 532 are controlled by the processor(s) 512, for example. In various implementations, the defibrillation electrodes 534 are connected to defibrillation leads 536. The defibrillation leads 536 are connected to a defibrillation port 538, in implementations. According to various examples, the defibrillation leads 536 are removable from the defibrillation port 538. For example, the defibrillation leads 536 are plugged into the defibrillation port 538.

The defibrillation electrodes 534 may include identifier data 554B that may include unique identifiers for the different electrodes that may be used to uniquely track the uses for each of the defibrillation electrodes and thereby determine the useful life and/or end of life for each defibrillation electrode as described herein.

In various implementations, the processor(s) 512 is operably connected to one or more transceivers 540 that transmit and/or receive data over one or more communication networks 542. For example, the transceiver(s) 540 includes a network interface card (NIC), a network adapter, a local area network (LAN) adapter, or a physical, virtual, or logical address to connect to the various external devices and/or systems. In various examples, the transceiver(s) 540 includes any sort of wireless transceivers capable of engaging in wireless communication (e.g., radio frequency (RF) communication). For example, the communication network(s) 542 includes one or more wireless networks that include a 3rd Generation Partnership Project (3GPP) network, such as a Long-Term Evolution (LTE) radio access network (RAN) (e.g., over one or more LE bands), a New Radio (NR) RAN (e.g., over one or more NR bands), or a combination thereof. In some cases, the transceiver(s) 540 includes other wireless modems, such as a modem for engaging in WI-FI®, WIGIG®, WIMAX®, BLUETOOTH®, or infrared communication over the communication network(s) 542.

The defibrillator 500 is configured to transmit and/or receive data (e.g., ECG data, impedance data, data indicative of one or more detected heart rhythms of the individual 508, data indicative of one or more defibrillation shocks administered to the individual 508, etc.) with one or more external devices 544 via the communication network(s) 542. The external devices 544 include, for instance, mobile devices (e.g., mobile phones, smart watches, etc.), Internet of Things (IOT) devices, medical devices, computers (e.g., laptop devices, servers, etc.), or any other type of computing device configured to communicate over the communication network(s) 542. In some examples, the external device(s) 544 is located remotely from the defibrillator 500, such as at a remote clinical environment (e.g., a hospital). According to various implementations, the processor(s) 512 causes the transceiver(s) 540 to transmit data to the external device(s) 544. In some cases, the transceiver(s) 540 receives data from the external device(s) 544 and the transceiver(s) 540 provide the received data to the processor(s) 512 for further analysis. In some examples, the external device(s) 544 may include the asset manager 112 and/or asset manager 114 of FIG. 1.

In various implementations, the external defibrillator 500 also includes a housing 546 that at least partially encloses other elements of the external defibrillator 500. For example, the housing 546 encloses the detection circuit 510, the processor(s) 512, the memory 514, the charging circuit 522, the transceiver(s) 540, or any combination thereof. In some cases, the input device(s) 518 and output device(s) 520 extend from an interior space at least partially surrounded by the housing 546 through a wall of the housing 546. In various examples, the housing 546 acts as a barrier to moisture, electrical interference, and/or dust, thereby protecting various components in the external defibrillator 500 from damage.

In some implementations, the external defibrillator 500 is an automated external defibrillator (AED) operated by an untrained user (e.g., a bystander, layperson, etc.) and can be operated in an automatic mode. In automatic mode, the processor(s) 512 automatically identifies a rhythm in the ECG signal, decides whether to administer a defibrillation shock, charges the capacitor(s) 530, discharges the capacitor(s) 530, or any combination thereof. In some cases, the processor(s) 512 controls the output device(s) 520 to output (e.g., display) a simplified user interface to the untrained user. For example, the processor(s) 512 refrains from causing the output device(s) 520 to display a waveform of the ECG signal and/or the impedance signal to the untrained user, in order to simplify operation of the external defibrillator 500.

In some examples, the external defibrillator 500 is a monitor-defibrillator utilized by a trained user (e.g., a user, an emergency responder, etc.) and can be operated in a manual mode or the automatic mode. When the external defibrillator 500 operates in manual mode, the processor(s) 512 cause the output device(s) 520 to display a variety of information that is relevant to the trained user, such as waveforms indicating the ECG data and/or impedance data, notifications about detected heart rhythms, and the like.

FIG. 6 illustrates an example of a capnography system 600 configured to perform various functions described herein, according to at least one example. FIG. 6 shows a portion of a patient 604, with an airway opening 606 and one of their lungs indicated as 118. A capnography system 600 according to one example is shown, which is configured to be used together with a ventilation system 602 according to implementations. In some implementations, the components of the capnography system 600 and system 602 overlap, for better cooperation. The capnography system 600 and/or the ventilation system 602 may either and/or both include an identifier 660 that may be read by a monitoring component (e.g., monitoring circuit 650) to provide indications of use to the external device(s) 658 which may include the asset manager 112 and/or asset manager 114.

Ventilation system 602 includes a gas source 608. The gas source 608 can be oxygen, air, a mixture thereof, other combinations of gases, etc. Gas source 608 can be configured to expel repeated bursts of the gas 612 through a gas source tube 610. For expelling, gas source 608 is mechanized or manual. In some manual implementations, gas source 608 includes a bag that can be squeezed by a rescuer, each squeezing delivering one of the bursts.

A ventilation system according to one example includes and/or is configured to work with an airway tube. Examples of airway tubes include an endotracheal (ET) tubes, supraglottic airway laryngopharyngeal tubes, etc. In this description often an ET tube is shown, but that is by way of example and not of limitation, plus aspects of its description for purposes of this document apply also to other types of airway tubes.

Ventilation system 602 also includes an endotracheal (ET) tube 622, of which two portions are shown in different scales, and connected by three dots. In particular, patient 604 is intubated by ET tube 622, and a first portion of ET tube 622 has been inserted through airway opening 606 into the trachea of patient 604. As such, a first end 624 of ET tube 622 is thus brought close to lung 626.

ET tube 622 defines an air path that communicates with gas source tube 610 and gas source 608. As such, when ET tube 622 is thus inserted in the patient's airway, it can be configured so as to guide the bursts 628 of gas as artificial inhalations to lung 626 of patient 604. Gas expulsion by gas source 608 results in a pressure difference between the interior of ET tube 622 and the atmosphere, slightly stretching ET tube 622. The pressure in the interior of ET tube 622 is referred to as the patient's airway pressure and is also closely related to the patient's intrathoracic pressure.

Additional components is coupled between ET tube 622 and gas source 608, in a manner that preserves and accommodates the air path. Such components includes adapters, fittings, valves, etc. Coupling can happen because the components are usually tubular, and circular, and employ a male-female matching configuration.

In the example of FIG. 6 such a component is an airway adapter 616, which is configured to be coupled between gas source 608 and ET tube 622. Airway adapter 616 has a first end 618 that is coupled to a second end 620 of ET tube 622. Airway adapter 616 also has a second end 614 that is coupled to gas source tube 610. Airway adapter 616 has a hollow interior, so as to accommodate the air path when it is thus coupled.

Capnograph system 600 can be configured to detect carbon dioxide in exhalations of patient 604, and also a pressure in the air path. Capnograph system 600 includes a capnography module 636 that has a carbon dioxide detector 642. Carbon dioxide detector 642 can be configured to generate a carbon dioxide signal 652 responsive to an amount of carbon dioxide detected within the air path of ventilation system 602.

Capnograph system 600 also includes a monitoring circuit 650 that is distinct from carbon dioxide detector 642. Monitoring circuit 650 can be configured to detect a pressure in the air path. Monitoring circuit 650 has a processing component 648 within capnography module 636, and distinct from carbon dioxide detector 642. In fact, in some implementations, monitoring circuit 650 is wholly included within capnography module 636, while in other implementations not necessarily. Processing component 648 can be configured to generate a pressure signal 654 responsive to the pressure detected in the air path by the monitoring circuit 650. Pressure signal 654, alone or in combination with other signals such as carbon dioxide signal 652, is used to detect spontaneous breaths of the patient.

In some examples, capnography module 636 communicates with the air path by means of a side tube 630, which can be configured to be coupled between airway adapter 616 and capnography module 636. In fact, airway adapter 616 is interposed in the air path for the purpose of providing the opportunity of side tube 630 to access the air path, for sampling the gases and the pressure therein. The gases include a mixture of gases expelled by gas source 608 as bursts, and also from patient 604 as exhalations. For operation, side tube 630 is passed through an opening 632 in a housing 634 of capnography module 636, or of a monitor that houses capnography module 636, a monitor-defibrillator system, etc. In such configurations, capnography module 636 can be characterized as a side stream capnograph.

In some implementations, capnography module 636 includes a cuvette 638, which is a small chamber. Side tube 630 can be coupled to cuvette 638. Capnography module 636 also includes a pump 644, which is configured to draw gas from the air path into cuvette 638. This way, gases in the air path can be sampled while in cuvette 638. After that, the sampled gases can be disposed of via an exhaust tube 646. It will be understood that pump 644 withdraws, for sampling the vertical column of the air path, relatively little gas compared to what is needed for ventilating the patient. In addition, as long as pump 644 withdraws gas at a constant rate, that will not mask the transient nature of a peak that is intended to be detected.

In such implementations, a light source 640 in capnography module 636 illuminates the interior of cuvette 638, and carbon dioxide detector 642 detects an amount of the carbon dioxide within cuvette 638, by measuring how much light from light source 640 reaches it. In some implementations, light source 640 emits infrared (IR) light.

As mentioned above, pressure signal 654, alone or in combination with other signals such as carbon dioxide signal 652, is used by a processor to detect spontaneous breaths of the patient according to implementations among other physiological parameters, including end-tidal carbon dioxide or other respiratory parameters.

In various implementations, the processing component 648 is operably connected to one or more transceivers that transmit and/or receive the carbon dioxide signal 652, the pressure signal 654, and other signals over one or more communication networks 656. For example, the transceiver(s) includes a network interface card (NIC), a network adapter, a local area network (LAN) adapter, or a physical, virtual, or logical address to connect to the various external devices and/or systems. In various examples, the transceiver(s) includes any sort of wireless transceivers capable of engaging in wireless communication (e.g., radio frequency (RF) communication). For example, the communication network(s) 656 includes one or more wireless networks that include a 3rd Generation Partnership Project (3GPP) network, such as a Long-Term Evolution (LTE) radio access network (RAN) (e.g., over one or more LE bands), a New Radio (NR) RAN (e.g., over one or more NR bands), or a combination thereof. In some cases, the transceiver(s) includes other wireless modems, such as a modem for engaging in WI-FI®, WIGIG®, WIMAX®, BLUETOOTH®, or infrared communication over the communication network(s) 656.

The capnography system 600 is configured to transmit and/or receive data (e.g., capnograph data with one or more external devices 658 via the communication network(s) 656. The external devices 658 include, for instance, mobile devices (e.g., mobile phones, smart watches, etc.), Internet of Things (IOT) devices, medical devices, computers (e.g., laptop devices, servers, etc.), or any other type of computing device configured to communicate over the communication network(s) 656. In some examples, the external device(s) 658 is located remotely from the capnography system 600, such as at a remote clinical environment (e.g., a hospital). According to various implementations, the processing component 648 causes the transceiver(s) to transmit data to the external device(s) 658. In some cases, the transceiver(s) receives data from the external device(s) 658 and the transceiver(s) provide the received data to the processing component 648 for further analysis, including analysis of reliability of the data for use as described herein.

### Example Clauses

A. A system for managing lifespans of medical device accessories, the system including: an accessory associated with a unique identifier; a medical device configured to: couple with the accessory; and determine use data for the accessory when coupled with the accessory, the use data indicating a usage history of the accessory; and an asset manager communicably coupled with the medical device, the asset manager being configured to: receive the use data from the medical device; determine an expected expiration of the accessory by analyzing the use data; and generate a notification indicating the expected expiration.
B. The system of clause A, wherein the asset manager is configured to receive the unique identifier from the medical device, and wherein the medical device is configured to access the unique identifier from a memory device coupled to the accessory when the accessory is coupled to the medical device.
C. The system of clause A or B, wherein the unique identifier is indicated by a visual identifier displayed on the accessory, and wherein the medical device is further configured to identify the unique identifier by detecting, using a camera, the visual identifier displayed on the accessory.
D. The system of any of clauses A to C, wherein the unique identifier includes a code encoded in a radio frequency identification (RFID) tag coupled to the accessory, and wherein the medical device is further configured to identify the unique identifier by detecting, using a transceiver, the code from the RFID tag.
E. The system of any of clauses A to D, wherein the accessory is coupled to the medical device via a fused connection, wherein the asset manager is configured to output the notification to the medical device, the notification indicating that the expected expiration has elapsed, and wherein the medical device is configured to disrupt the fused connection in response to receiving the notification.
F. The system of any of clauses A to E, wherein the use data is determined using: a use time for the accessory; a temperature of the accessory; a volume of fluid conveyed by the accessory; a number of use cycles completed by the accessory; a number of inflations completed by the accessory; a number of times the accessory has connected to external devices; an amount of energy output by the accessory; or an amount of charges delivered by the accessory.
G. The system of any of clauses A to F, wherein the asset manager includes a cloud-based service configured to communicate with additional medical devices and computing devices of a service provider.
H. The system of any of clauses A to G, wherein the asset manager is further configured to: generate a report indicating the additional medical devices or additional accessories that have been coupled with the additional medical devices, the report including expected expirations of the additional medical devices or additional accessories; and transmit the report to a computing device among the computing devices of the service provider.
I. The system of any of clauses A to H, wherein the asset manager is configured to generate order data including item quantities and prices for accessories in response to the expected expiration.
J. The system of clause I, wherein the order data includes item quantities and prices for a predetermined time period.
K. The system of any of clauses A to J, wherein the asset manager is configured to generate an order for submission with a supplier to replace the accessory in response to the expected expiration.
L. The system of any of clauses A to K, wherein the asset manager is configured to generate, in response to the expected expiration having elapsed, a message for an account associated with the medical device, the message including the expected expiration and generated.
M. The system of any of clauses A to L, wherein the accessory is coupleable to additional medical devices, and wherein the asset manager is further configured to: store the use data and the unique identifier in an entry of a database; receive, from the additional medical devices, additional use data associated with the unique identifier, the additional use data indicating usage of the accessory with the additional medical devices; and update the entry to reflect the additional use data.
N. The system of any of clauses A to M, wherein the asset manager includes a computing component on-board the medical device, the computing component including a processor and a non-transitory computer-readable medium storing instructions thereon that, when executed by the processor, cause the processor to determine the expected expiration of the accessory.
O. A cloud-based medical device accessory management system including: medical devices; accessories coupleable to the medical devices and being associated, respectively, with unique identifiers; and a cloud-based asset manager communicably coupled with the medical devices, the cloud-based asset manager being configured to: receive accessory use data from the medical devices associated, respectively, with the unique identifiers; determine expected expirations of the accessories by analyzing the accessory use data and the unique identifiers associated with the accessories; and generate a notification indicating the expected expirations.
P. The cloud-based medical device accessory management system of clause O, wherein the medical devices and accessories are associated with a service provider.
Q. The cloud-based medical device accessory management system of clause O or P, wherein the cloud-based asset manager is configured to: identify a subset of the accessories associated with a subset of the expected expirations that are within a range of expected expirations; and generate order data including item quantities and prices for the subset of the accessories.
R. The cloud-based medical device accessory management system of clause Q, wherein the order data includes item quantities and prices for replacing the subset of accessories.
S. The cloud-based medical device accessory management system of clause Q or R, wherein the cloud-based asset manager is configured to generate an order for submission with a supplier to replace the subset of the accessories.
T. The cloud-based medical device accessory management system of any of clauses O to S, wherein the cloud-based asset manager is configured to generate a message for an account associated with the medical devices, the message including the expected expirations.
U. A medical device, including: a first communication interface configured to connect with an accessory; a second communication interface configured to connect with a cloud-based management system; a processor; and a non-transitory computer-readable medium having instructions stored thereon that, when executed by the processor, cause the processor to: in response to the first communication interface connecting with the accessory, determine an identity associated with the accessory; determine use data indicating a usage of the accessory; determine, by analyzing the use data, an expected expiration of the connected accessory; and generate a message in response to determining the expected expiration.
V. The medical device of clause U, wherein the instructions to determine the identity of the accessory include instructions to read a memory device coupled to the accessory. W. The medical device of clause U or V, further including: a camera configured to capture an image depicting a visual identifier displayed on the accessory, wherein the instructions to determine the identity of the accessory include analyzing the visual identifier.
X. The medical device of any of clauses U to W, further including: a radio frequency identification (RFID) sensor, wherein the instructions to determine the identity of the accessory include instructions to read a code detected by the RFID sensor from an RFID tag associated with the accessory.
Y. The medical device of any of clauses U to X, wherein the first communication interface includes a fused connection, wherein the instructions further include determining the expected expiration has expired, and wherein the fused connection is configured to disconnect from the accessory in response to determining the expected expiration has elapsed.
Z. The medical device of any of clauses U to Y, wherein the use data is determined using: a use time for the accessory; a temperature of the accessory; a volume of fluid conveyed by the accessory; a number of use cycles completed by the accessory; a number of inflations completed by the accessory; a number of connections between the accessory and other medical devices; a type of the medical device; or an amount of delivered charges of the accessory.
AA. The medical device of any of clauses U to Z, wherein the cloud-based management system is configured to generate a report of medical devices and accessories for a service provider, the report including expected expirations for the medical device or accessories.
AB. The medical device of any of clauses U to AA, wherein the medical device includes: an ultrasound device; an oximetry device; a defibrillation device; a capnography device; or a treatment device configured to provide a treatment to a patient.
AC. A method including: determining a unique identifier associated with an accessory coupled to a medical device; determining, by the medical device, use data for the accessory when the accessory is coupled to the medical device, wherein the use data includes indications of use for the accessory; conveying, by the medical device and to a cloud-based asset manager, the use data for the accessory and the unique identifier; determining, using the use data, an expected expiration for the accessory; and generating, in response to determining the expected expiration, a report including the unique identifier and the expected expiration for the accessory.
AD. The method of clause AC, wherein the cloud-based asset manager stores and updates use data for the accessory in response to the use data from the medical device.
AE. The method of clause AD, wherein determining the expected expiration includes the medical device determining the expected expiration using lifetime use data stored at the cloud-based asset manager, the lifetime use data including aggregated use data for the accessory over a lifespan of the accessory.
AF. The method of any of clauses AC to AE, further including determining conditions for expiration using categories of use data, the categories of use data describing metrics for use of the accessory.
AG. The method of clause AF, wherein determining the expected expiration is further performed using the conditions for expiration.
AH. The method of any of clauses AC to AG, wherein determining the expected expiration includes inputting the use data from the medical device and aggregated use data associated with the accessory from the cloud-based asset manager to a machine learned model trained using compiled use data for accessories labeled with expiration data for the accessories.
AI. The method of clause AH, wherein determining the expected expiration includes the medical device inputting the use data and the aggregated use data into the machine learned model, the machine learned model hosted on a component of the medical device.

While the example clauses described above are described with respect to one particular implementation, it should be understood that, in the context of this document, the content of the example clauses can also be implemented via a method, device, system, computer-readable medium, and/or another implementation. Additionally, any of examples A-AI may be implemented alone or in combination with any other one or more of the examples A-AI.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of, or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the implementation to the specified elements, steps, ingredients, or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is warranted, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member is referred to and claimed individually or in any combination with other members of the group or other elements found herein. It is anticipated that one or more members of a group is included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system for managing lifespans of medical device accessories, the system comprising:
an accessory associated with a unique identifier;
a medical device configured to:
couple with the accessory; and
determine use data for the accessory when coupled with the accessory,
the use data indicating a usage history of the accessory; and
an asset manager communicably coupled with the medical device, the asset manager being configured to:
receive the use data from the medical device;
determine an expected expiration of the accessory by analyzing the use data; and
generate a notification indicating the expected expiration.

2. The system of claim 1, wherein the asset manager is configured to receive the unique identifier from the medical device, and
wherein the medical device is configured to access the unique identifier from a memory device coupled to the accessory when the accessory is coupled to the medical device.

3. The system of claim 1, wherein the accessory is coupled to the medical device via a fused connection,
wherein the asset manager is configured to output the notification to the medical device, the notification indicating that the expected expiration has elapsed, and
wherein the medical device is configured to disrupt the fused connection in response to receiving the notification.

4. The system of claim 1, wherein the asset manager comprises a cloud-based service configured to communicate with additional medical devices and computing devices of a service provider.

5. The system of claim 4, wherein the asset manager is further configured to:
generate a report indicating the additional medical devices or additional accessories that have been coupled with the additional medical devices, the report comprising expected expirations of the additional medical devices or additional accessories; and
transmit the report to a computing device among the computing devices of the service provider.

6. The system of claim 1, wherein the asset manager is configured to generate, in response to the expected expiration having elapsed, a message for an account associated with the medical device, the message comprising the expected expiration and generated.

7. The system of claim 1, wherein the accessory is coupleable to additional medical devices, and
wherein the asset manager is further configured to:
store the use data and the unique identifier in an entry of a database;
receive, from the additional medical devices, additional use data associated with the unique identifier, the additional use data indicating usage of the accessory with the additional medical devices; and
update the entry to reflect the additional use data.

8. The system of claim 1, wherein the asset manager comprises a computing component on-board the medical device, the computing component comprising a processor and a non-transitory computer-readable medium storing instructions thereon that, when executed by the processor, cause the processor to determine the expected expiration of the accessory.

9. A medical device, comprising:
a first communication interface configured to connect with an accessory;
a second communication interface configured to connect with a cloud-based management system;
a processor; and
a non-transitory computer-readable medium having instructions stored thereon that, when executed by the processor, cause the processor to:
in response to the first communication interface connecting with the accessory, determine an identity associated with the accessory;
determine use data indicating a usage of the accessory;
determine, by analyzing the use data, an expected expiration of the connected accessory; and
generate a message in response to determining the expected expiration.

10. The medical device of claim 9, wherein the instructions to determine the identity of the accessory comprise instructions to read a memory device coupled to the accessory.

11. The medical device of claim 9, further comprising:
a camera configured to capture an image depicting a visual identifier displayed on the accessory,
wherein the instructions to determine the identity of the accessory comprise analyzing the visual identifier.

12. The medical device of claim 9, further comprising:
a radio frequency identification (RFID) sensor,
wherein the instructions to determine the identity of the accessory comprise instructions to read a code detected by the RFID sensor from an RFID tag associated with the accessory.

13. The medical device of claim 9, wherein the first communication interface comprises a fused connection,
wherein the instructions further comprise determining the expected expiration has expired, and
wherein the fused connection is configured to disconnect from the accessory in response to determining the expected expiration has elapsed.

14. The medical device of claim 9, wherein the use data is determined using:
a use time for the accessory;
a temperature of the accessory;
a volume of fluid conveyed by the accessory;
a number of use cycles completed by the accessory;
a number of inflations completed by the accessory;
a number of connections between the accessory and other medical devices;
a type of the medical device; or
an amount of delivered charges of the accessory.

15. The medical device of claim 9, wherein the cloud-based management system is configured to generate a report of medical devices and accessories for a service provider, the report comprising expected expirations for the medical device or accessories.

16. A method comprising:
determining a unique identifier associated with an accessory coupled to a medical device;
determining, by the medical device, use data for the accessory when the accessory is coupled to the medical device, wherein the use data comprises indications of use for the accessory;
conveying, by the medical device and to a cloud-based asset manager, the use data for the accessory and the unique identifier;
determining, using the use data, an expected expiration for the accessory; and
generating, in response to determining the expected expiration, a report comprising the unique identifier and the expected expiration for the accessory.

17. The method of claim 16, wherein the cloud-based asset manager stores and updates use data for the accessory in response to the use data from the medical device.

18. The method of claim 17, wherein determining the expected expiration comprises the medical device determining the expected expiration using lifetime use data stored at the cloud-based asset manager, the lifetime use data comprising aggregated use data for the accessory over a lifespan of the accessory.

19. The method of claim 16, further comprising determining conditions for expiration using categories of use data, the categories of use data describing metrics for use of the accessory, and wherein determining the expected expiration is further performed using the conditions for expiration.

20. The method of claim 16, wherein determining the expected expiration comprises inputting the use data from the medical device and aggregated use data associated with the accessory from the cloud-based asset manager to a machine learned model trained using compiled use data for accessories labeled with expiration data for the accessories.
